(19) United States
(12) Patent Application Publication
(10) Pub. No.: US 2026/0177555 A1
(43) Pub. Date: Jun. 25, 2026

(54) CARRY-OVER FREE METHOD FOR SEMAGLUTIDE QUANTITATION

(71) Applicant: SHIMADZU CORPORATION, Kyoto-shi (JP)

(72) Inventors: Nitin SHUKLA, Mumbai (IN); Nitish SURYAWANSHI, Mumbai (IN); Sujit PATIL, Mumbai (IN); Jitendra KELKAR, Mumbai (IN); Pratap RASAM, Mumbai (IN)

(73) Assignee: SHIMADZU CORPORATION, Kyoto-shi (JP)

(21) Appl. No.: 19/429,897

(22) Filed: Dec. 22, 2025

(30) Foreign Application Priority Data
Dec. 24, 2024 (IN) 202411102674

(51) Int. Cl. G01N 33/68; G01N 1/34; G01N 30/02; G01N 30/88

(52) U.S. Cl. CPC G01N 33/68; G01N 1/34; G01N 30/88; G01N 2030/027; G01N 2030/8831; G01N 2333/605

(57) ABSTRACT

The present disclosure relates to a method of separating, purifying, detecting, measuring, or analyzing a sample using a liquid chromatography.

### BACKGROUND

Analysis of synthetic peptides such as Semaglutide using high-performance liquid chromatography (HPLC) followed by mass spectrometry (MS) are extremely challenging, due to, among other reasons, the nonspecific adsorption that is observed in HPLC autosampler parts and analysis column. This results in significant carryover of materials analyses, restricting the analytical methods to achieve the desired limit of quantitation (LOQ). To get rid of the contamination from previous injection, the conventional approach involves use of high affinity solvents such as water, organic solvents, harsh solvents or harsh aqueous solutions with specific pH, mixture of solvents etc. however, with one at a time approach.

Additionally, analyzing higher concentration of such molecules results in even higher carryover. To avoid this situation, many conventional LC-MS/MS methods cover a limited calibration range for quantitation. A method with short calibration range has a limited applicability for drug formulations with wide label claim or dosage concentrations when it comes to its bioavailability and bioequivalence (BABE) study in drug development. This brings us to a need of having a novel, sensitive, accurate, and robust analytical method for quantitation of synthetic peptide such as semaglutide using LC-MS/MS with no carryover of contaminants from previous injections and wide dynamic range, which is applicable to drug formulations with a broad range of dosage concentrations.

### SUMMARY

In one aspect, the present disclosure provides a method of detecting, measuring, or analyzing a sample using liquid chromatography comprising rinsing a sampling means of a liquid chromatography system with a first rinsing solution. In some embodiments, the method further comprises rinsing the sampling means with a second rinsing solution. In one aspect, the present disclosure provides a composition for detecting, measuring, or analyzing a sample using liquid chromatography described herein.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 shows a diagram of internal rinsing of needle with rinse solution (R0, R1, and R2).

FIG. 2 shows a diagram of internal and external rinsing.

FIG. 3 shows a structure of semaglutide.

FIG. 4 shows an analytical condition.

FIG. 5 shows chromatograms for blank, LLOQ, ULOQ, and calibration curve.

FIG. 6 shows diagram of sample analysis.

FIG. 7 shows system precision and specificity.

FIG. 8 shows linearity range.

FIG. 9 shows accuracy and precision results.

FIG. 10 shows chromatographic overlay of blank, LLOQ, ULOQ to depict no carryover after ULOQ sample.

FIG. 11 shows blank analysis result.

FIG. 12 shows a method report.

FIG. 13 shows chromatography results of semaglutide.

FIG. 14 shows chromatography results.

### DETAILED DESCRIPTION

Hereinafter, the present disclosure will be described in more detail to help the understanding of the present disclosure.

As used herein and unless otherwise indicated, “% v/v” refers to a volume percent based on a total volume of a reference unless otherwise explained.

When the term “about” is used, it is used to mean a certain effect or result can be obtained within a certain tolerance, and the skilled person knows how to obtain the tolerance. When the term “about” is used in describing a value or an end-point of a range, the disclosure should be understood to include the specific value or end-point referred to. In one aspect, the term “about” means plus or minus 20, 19, 18, 17, 16, 15, 14, 13, 12, 11, 10, 9, 8, 7, 6, 5, 4, 3, 2, or 1% of the numerical value of the number with which it is being used.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but can include other elements not expressly listed or inherent to such process, method, article, or apparatus.

As used herein, the phrase “sampling means” may include any hardware, components, parts, or devices that can be used to draw a sample to be analyzed using liquid chromatography, examples of which include a needle, a pipette, a syringe, a tube, and equivalents thereof.

The transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified materials or steps and those that do not materially affect the basic and novel characteristic(s) of the claimed embodiment. A “consisting essentially of” claim occupies a middle ground between closed claims that are written in a “consisting of” format and fully open claims that are drafted in a “comprising” format. Optional additives as defined herein, at a level that is appropriate for such additives, and minor impurities are not excluded from a composition by the term “consisting essentially of”.

Further, unless expressly stated to the contrary, “or” and “and/or” refers to an inclusive and not to an exclusive. For example, a condition A or B, or A and/or B, is satisfied by any one of the following: A is true (or present) and B is false (or not present), A is false (or not present) and B is true (or present), and both A and B are true (or present).

The use of “a” or “an” to describe the various elements and components herein is merely for convenience and to give a general sense of the disclosure. This description should be read to include one or at least one and the singular also includes the plural unless it is obvious that it is meant otherwise.

As used herein, the term “chromatography” refers to a process in which a chemical mixture carried by a liquid or gas is separated into components as a result of differential distribution of the chemical entities as they flow around or over a stationary liquid or solid phase.

As used herein, the term “liquid chromatography” or “LC” refers a process of selective retardation of one or more components of a fluid solution as the fluid uniformly percolates through a column of a finely divided substance, or through capillary passageways.

The retardation results from the distribution of the components of the mixture between one or more stationary phases and the bulk fluid, (i.e., mobile phase), as this fluid moves relative to the stationary phase(s). Examples of “liquid chromatography” include reverse phase liquid chromatography (RPLC), high performance liquid chromatography (HPLC), and turbulent flow liquid chromatography (TFLC) (sometimes known as high turbulence liquid chromatography (HTLC) or high throughput liquid chromatography.

As used herein, the term “mass spectrometry” or “MS” refers to an analytical technique to identify compounds by their mass. MS refers to methods of filtering, detecting, and measuring ions based on their mass-to-charge ratio, or “m/z”. MS technology generally includes (1) ionizing the compounds to form charged compounds; and (2) detecting the molecular weight of the charged compounds and calculating a mass-to-charge ratio. The compounds may be ionized and detected by any suitable means. A “mass spectrometer” generally includes an ionizer, a mass analyzer, and an ion detector. In general, one or more molecules of interest are ionized, and the ions are subsequently introduced into a mass spectrometric instrument where, due to a combination of magnetic and electric fields, the ions follow a path in space that is dependent upon mass (“m”) and charge (“z”).

In one aspect, the present disclosure provides a method of separating, purifying, detecting, measuring, or analyzing a sample. In some embodiments, the method uses a liquid chromatography. In some embodiments, the method comprises rinsing a sampling means of a liquid chromatography with a first rinsing solution.

In some embodiments, the method comprises separating or purifying the sample or a component, such as a protein, of the sample. In some embodiments, the sample or the protein may be semaglutide.

In some embodiments, the method comprises detecting, measuring, or analyzing a presence of absence, an amount, a concentration, or a mass of the sample, or a component, such as a protein, and therein. In some embodiments, the sample or the protein may be semaglutide.

In some embodiments, the detecting may comprise using a detector to identify and/or quantify the compounds separated in the column. In some embodiment, the compound comprises sample or a component, such as a protein, of the sample. In some embodiments, the sample or the protein may be semaglutide.

“Sample” means a quantity of material from a biological, environmental, medical, or patient source in which separation, purification, detection, measurement, or analysis is sought. On the one hand it is meant to include a specimen or culture (e.g., microbiological cultures). On the other hand, it is meant to include both biological and environmental samples. A sample may include a specimen of synthetic origin. Environmental samples include environmental material, such as surface matter, soil, water and industrial samples, as well as samples obtained from food and dairy processing instruments, apparatus, equipment, utensils, disposable and non-disposable items. The sample may be obtained from a source, including, but not limited to, whole blood, serum, plasma, urine, saliva, sweat, fecal matter, tears, intestinal fluid, mucous membrane samples, lung tissue, tumors, transplanted organs, fetus, and/or other sources. The samples may be from an animal, including human, fluid, solid (e.g., stool) or tissue. The samples may include materials taken from a patient including, but not limited to cultures, blood, saliva, cerebral spinal fluid, pleural fluid, milk, lymph, sputum, semen, needle aspirates, and the like. In some embodiments, the sample comprises semaglutide contained in a biological sample described above. In some embodiments, the sample consists of semaglutide contained in a biological sample. In some embodiments, the sample is semaglutide contained in a biological sample.

In some embodiments, the first rinsing solution comprises one or more, two or more, three or more, or four or more solvents from the group consisting of trifluoroacetic acid, ammonia, formic acid, acetonitrile, methanol, water, and 2-propanol. In some embodiments, the first rinsing solution comprises trifluoroacetic acid. In some embodiments, the first rinsing solution comprises ammonia. In some embodiments, the first rinsing solution comprises formic acid. In some embodiments, the first rinsing solution comprises acetonitrile. In some embodiments, the first rinsing solution comprises methanol. In some embodiments, the first rinsing solution comprises water. In some embodiments, the first rinsing solution comprises 2-propanol. In some embodiments, the first rinsing solution further comprises at least one selected from the group consisting of acetonitrile, methanol, water, and 2-propanol. In some embodiments, the first rinsing solution further comprises acetonitrile. In some embodiments, the first rinsing solution further comprises methanol. In some embodiments, the first rinsing solution further comprises water. In some embodiments, the first rinsing solution further comprises 2-propanol. In some embodiments, the first rinsing solution comprises aqueous solvent and organic solvents.

In some embodiments, the first rinsing solution comprises trifluoroacetic acid. In some embodiments, the first rinsing solution comprises 1% v/v trifluoroacetic acid. In some embodiments, the first rinsing solution comprises at least 0.1%, 0.5%, 1%, 2%, 3%, 4%, 5%, 6%, 7%, 8%, 9%, 10%, 15%, 20%, 30%, 40%, or 50% v/v trifluoroacetic acid. In some embodiments, the first rinsing solution comprises at most 50%, 45%, 40%, 35%, 30%, 25%, 20%, 15%, 10%, 9%, 8%, 7%, 6%, 5%, 4%, 3%, 2%, 1.5%, 1%, or 0.5% v/v trifluoroacetic acid. In some embodiments, the first rinsing solution comprises from about 0.1% to about 50%, from about 0.1% to about 40%, from about 0.1% to about 30%, from about 0.1% to about 20%, from about 0.1% to about 10%, from about 0.1% to about 5%, from about 0.1% to about 3%, from about 0.1% to about 1%, from about 0.5% to about 50%, from about 0.5% to about 40%, from about 0.5% to about 30%, from about 0.5% to about 20%, from about 0.5% to about 10%, from about 0.5% to about 5%, from about 0.5% to about 3%, from about 0.5% to about 1.5%, from about 0.5% to about 1%, from about 1% to about 50%, from about 1% to about 40%, from about 1% to about 30%, from about 1% to about 20%, from about 1% to about 10%, from about 1% to about 5%, from about 1% to about 4%, from about 1% to about 3%, or from about 1% to about 2% v/v trifluoroacetic acid.

In some embodiments, the method comprises 1% v/v of the first rinsing solution comprising solvent from the group consisting of trifluoroacetic acid, ammonia, formic acid, acetonitrile, methanol, water, and 2-propanol. In some embodiments, the method comprises at least 0.1%, 0.5%, 1%, 2%, 3%, 4%, 5%, 6%, 7%, 8%, 9%, 10%, 15%, 20%, 30%, 40%, or 50% v/v of the first rinsing solution comprising solvent from the group consisting of trifluoroacetic acid, ammonia, formic acid, acetonitrile, methanol, water, and 2-propanol. In some embodiments, the method comprises at most 50%, 45%, 40%, 35%, 30%, 25%, 20%, 15%, 10%, 9%, 8%, 7%, 6%, 5%, 4%, 3%, 2%, 1.5%, 1%, or 0.5% v/v of the first rinsing solution comprising solvent from the group consisting of trifluoroacetic acid, ammonia, formic acid, acetonitrile, methanol, water, and 2-propanol. In some embodiments, the method comprises from about 0.1% to about 50%, from about 0.1% to about 40%, from about 0.1% to about 30%, from about 0.1% to about 20%, from about 0.1% to about 10%, from about 0.1% to about 5%, from about 0.1% to about 3%, from about 0.1% to about 1%, from about 0.5% to about 50%, from about 0.5% to about 40%, from about 0.5% to about 30%, from about 0.5% to about 20%, from about 0.5% to about 10%, from about 0.5% to about 5%, from about 0.5% to about 3%, from about 0.5% to about 1.5%, from about 0.5% to about 1%, from about 1% to about 50%, from about 1% to about 40%, from about 1% to about 30%, from about 1% to about 20%, from about 1% to about 10%, from about 1% to about 5%, from about 1% to about 4%, from about 1% to about 3%, or from about 1% to about 2% v/v of the first rinsing solution comprising solvent from the group consisting of trifluoroacetic acid, ammonia, formic acid, acetonitrile, methanol, water, and 2-propanol.

In some embodiments, the first rinsing solution further comprises at least one selected from the group consisting of acetonitrile, methanol, water, and 2-propanol. In some embodiments, the first rinsing solution further comprises acetonitrile, methanol, water, and 2-propanol. In some embodiments, the first rinsing solution comprises equal volumes of acetonitrile, methanol, water, and 2-propanol. In some embodiments, the first rinsing solution comprises unequal volumes of acetonitrile, methanol, water, and 2-propanol.

In some embodiments, the rinsing the sampling means with a first rinsing solution comprises purging the first rinsing solution from the sampling means. In some embodiments, the rinsing the sampling means with the first rinsing solution further comprises rinsing an injection port.

In some embodiments, the first rinsing solution can be used as internal rinse. In some embodiments, the first rinsing solution can be used as external rinse. In some embodiments, the first rinsing solution can rinse an autosampler flow path.

In some embodiments, the method further comprises rinsing the sampling means with a second rinsing solution. In some embodiments, the second rinsing solution comprises one or more, two or more, three or more, or four or more solvents selected from the group consisting of N, N-dimethylsulfoxide (DMSO), Acetonitrile, methanol, trifluoracetic acid, formic acid & ammonium, and water. In some embodiments, the second rinsing solution comprises DMSO. In some embodiments, the second rinsing solution comprises Acetonitrile. In some embodiments, the second rinsing solution comprises methanol. In some embodiments, the second rinsing solution comprises trifluoracetic acid. In some embodiments, the second rinsing solution comprises formic acid and ammonium. In some embodiments, the second rinsing solution comprises formic acid. In some embodiments, the second rinsing solution comprises ammonium. In some embodiments, the second rinsing solution comprises water. In some embodiments, the second rinsing solution can be used to clean an external of the needle.

In some embodiments, the second rinsing solution comprises about 5% v/v DMSO. In some embodiments, the second rinsing solution comprise at least 0.1%, 0.5%, 1%, 2%, 3%, 4%, 5%, 6%, 7%, 8%, 9%, 10%, 15%, 20%, 30%, 40%, or 50% v/v DMSO. In some embodiments, the first rinsing solution comprises at most 50%, 45%, 40%, 35%, 30%, 25%, 20%, 15%, 10%, 9%, 8%, 7%, 6%, 5%, 4%, 3%, 2%, 1.5%, 1%, or 0.5% v/v DMSO. In some embodiments, the first rinsing solution comprises from about 0.1% to about 50%, from about 0.1% to about 40%, from about 0.1% to about 30%, from about 0.1% to about 20%, from about 0.1% to about 10%, from about 0.1% to about 5%, from about 0.1% to about 3%, from about 0.1% to about 1%, from about 0.5% to about 50%, from about 0.5% to about 40%, from about 0.5% to about 30%, from about 0.5% to about 20%, from about 0.5% to about 10%, from about 0.5% to about 5%, from about 0.5% to about 3%, from about 0.5% to about 1.5%, from about 0.5% to about 1%, from about 1% to about 50%, from about 1% to about 40%, from about 1% to about 30%, from about 1% to about 20%, from about 1% to about 10%, from about 1% to about 5%, from about 1% to about 4%, from about 1% to about 3%, or from about 1% to about 2%, from about 3% to about 50%, from about 3% to about 40%, from about 3% to about 30%, from about 3% to about 20%, from about 3% to about 10%, from about 3% to about 5%, or from about 3% to about 4% v/v DMSO.

In some embodiments, the rinsing the sampling means with the second rinsing solution comprises immersing a portion of the sampling means in the second rinsing solution. In some embodiments, the rinsing the sampling means with the second rinsing solution further comprises agitating or pumping the second rinsing solution while the portion of the sampling means remain immersed.

In some embodiments, the method further comprises loading a liquid chromatography column with the sample via the sampling means and/or performing liquid chromatography with the liquid chromatography column. In some embodiments, the method further comprises loading a liquid chromatography column with the sample via the sampling means. In some embodiments, the method further comprises performing liquid chromatography with the liquid chromatography column.

In some embodiments, the method further comprises performing mass spectrometry on an elute from the liquid chromatography. The elute from the liquid chromatography can be detected, measured or analyzed by other methods, such as a UV detection. In some embodiments, he elute from the liquid chromatography can be detected, measured or analyzed by other methods such as ultraviolet-visible light spectrophotometer (“UV detector”) using a photodiode array detector.

In some embodiments, the sample comprises a peptide or a protein. In some embodiments, the sample comprises a peptide or a protein contained in a biological sample. In some embodiments, the sample consists of peptide or protein. In some embodiments, the sample is a peptide contained in a biological sample. In some embodiments, the sample comprises a synthetic peptide or protein. In some embodiments, the sample comprises a synthetic peptide or protein contained in a biological sample. In some embodiments, the sample consisting of a synthetic peptide or protein contained in a biological sample. In some embodiments, the sample is a synthetic peptide or protein contained in a biological sample. In some embodiments, the sample comprises a non-synthetic, natural peptide or protein. In some embodiments, the sample comprises a natural peptide or protein contained in a biological sample. In some embodiments, the sample consisting of a natural peptide or protein contained in a biological sample. In some embodiments, the sample is a natural peptide or protein contained in a biological sample. In some embodiments, the sample comprises semaglutide.

In some embodiments, the liquid chromatography comprises high-performance liquid chromatography (HPLC). In some embodiments, the liquid chromatography comprises fast protein liquid chromatography (FPLC). In some embodiments, the liquid chromatography comprises liquid-liquid chromatography.

In some embodiments, a column conditioning solution comprises one or more solvents selected from the group consisting of DMSO, acetonitrile, methanol and formic acid. In some embodiments, the column conditioning solution comprises DMSO. In some embodiments, the column conditioning solution comprises acetonitrile. In some embodiments, the column conditioning solution comprises methanol. In some embodiments, the column conditioning solution comprises formic acid.

In some embodiments, the column conditioning solution comprises 0.5% v/v formic acid. In some embodiments, the column conditioning solution comprises 1% v/v formic acid in water. In some embodiments, the column conditioning solution comprises 1% v/v formic acid in methanol. In some embodiments, the column conditional solution comprises at least 0.1%, 0.5%, 1%, 2%, 3%, 4%, 5%, 6%, 7%, 8%, 9%, 10%, 15%, 20%, 30%, 40%, or 50% v/v formic acid. In some embodiments, the column conditional solution comprises at most 50%, 45%, 40%, 35%, 30%, 25%, 20%, 15%, 10%, 9%, 8%, 7%, 6%, 5%, 4%, 3%, 2%, 1.5%, 1%, or 0.5% v/v formic acid. In some embodiments, the column conditional solution comprises from about 0.1% to about 50%, from about 0.1% to about 40%, from about 0.1% to about 30%, from about 0.1% to about 20%, from about 0.1% to about 10%, from about 0.1% to about 5%, from about 0.1% to about 3%, from about 0.1% to about 1%, from about 0.5% to about 50%, from about 0.5% to about 40%, from about 0.5% to about 30%, from about 0.5% to about 20%, from about 0.5% to about 10%, from about 0.5% to about 5%, from about 0.5% to about 3%, from about 0.5% to about 1.5%, from about 0.5% to about 1%, from about 1% to about 50%, from about 1% to about 40%, from about 1% to about 30%, from about 1% to about 20%, from about 1% to about 10%, from about 1% to about 5%, from about 1% to about 4%, from about 1% to about 3%, or from about 1% to about 2%, from about 3% to about 50%, from about 3% to about 40%, from about 3% to about 30%, from about 3% to about 20%, from about 3% to about 10%, from about 3% to about 5%, or from about 3% to about 4% v/v formic acid.

In some embodiments, the column conditioning solution comprises 1.0% v/v DMSO. In some embodiments, the column conditional solution comprises at least 0.1%, 0.5%, 1%, 2%, 3%, 4%, 5%, 6%, 7%, 8%, 9%, 10%, 15%, 20%, 30%, 40%, or 50% v/v DMSO. In some embodiments, the column conditional solution comprises at most 50%, 45%, 40%, 35%, 30%, 25%, 20%, 15%, 10%, 9%, 8%, 7%, 6%, 5%, 4%, 3%, 2%, 1.5%, 1%, or 0.5% v/v DMSO. In some embodiments, the column conditional solution comprises from about 0.1% to about 50%, from about 0.1% to about 40%, from about 0.1% to about 30%, from about 0.1% to about 20%, from about 0.1% to about 10%, from about 0.1% to about 5%, from about 0.1% to about 3%, from about 0.1% to about 1%, from about 0.5% to about 50%, from about 0.5% to about 40%, from about 0.5% to about 30%, from about 0.5% to about 20%, from about 0.5% to about 10%, from about 0.5% to about 5%, from about 0.5% to about 3%, from about 0.5% to about 1.5%, from about 0.5% to about 1%, from about 1% to about 50%, from about 1% to about 40%, from about 1% to about 30%, from about 1% to about 20%, from about 1% to about 10%, from about 1% to about 5%, from about 1% to about 4%, from about 1% to about 3%, or from about 1% to about 2%, from about 3% to about 50%, from about 3% to about 40%, from about 3% to about 30%, from about 3% to about 20%, from about 3% to about 10%, from about 3% to about 5%, or from about 3% to about 4% / DMSO.

In some embodiments, the sample shows a significant amount of non-specific adsorption on liquid chromatography flow path. In some embodiments, the sample is soluble in DMSO. In some embodiments, the sample is soluble in the second rinsing solution.

In some embodiments, the composition described herein excludes at least one solvent selected from the group consisting of benzene, carbon disulphide, carbon tetrachloride, dichloromethane (DCM), methylene chloride, toluene, xylene, white spirit, acetone, and ethyl acetate. In some embodiments, the composition described herein contains less than 10, 9, 8, 7, 6, 5, 4, 3, 2, or 1% v/v of water, organic solvent, or at least one solvent selected from the group consisting of benzene, carbon disulphide, carbon tetrachloride, dichloromethane (DCM), methylene chloride, toluene, xylene, white spirit, acetone, and ethyl acetate.

In some embodiments, the method measures the sample at least 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or 1000 ng/mL. In some embodiments, the method measures the sample at most 10000, 9000, 8000, 7000, 6000, 5000, 4000, 3000, 2000, 1000, 950, 900, 850, 800, 750, 700, 650, 600, 550, 500, 450, 400, 350, 300, 250, 200, 150, 100, 90, 80, 70, 60, 50, 40, 30, 20, or 10 ng/mL. In some embodiment, the method measures the sample from about 0.01 to about 1500 ng/mL, from about 0.01 to about 1000 ng/mL, from about 0.01 to about 900 ng/mL, from about 0.01 to about 800 ng/mL, from about 0.01 to about 700 ng/mL, from about 0.01 to about 600 ng/mL, from about 0.01 to about 500 ng/mL, from about 0.01 to about 400 ng/mL, from about 0.01 to about 300 ng/mL, from about 0.01 to about 200 ng/mL, from about 0.01 to about 100 ng/mL, from about 0.01 to about 50 ng/mL, from about 0.1 to about 1500 ng/mL, from about 0.1 to about 1000 ng/mL, from about 0.1 to about 900 ng/mL, from about 0.1 to about 800 ng/mL, from about 0.1 to about 700 ng/mL, from about 0.1 to about 600 ng/mL, from about 0.1 to about 500 ng/mL, from about 0.1 to about 400 ng/mL, from about 0.1 to about 300 ng/mL, from about 0.1 to about 200 ng/mL, from about 0.1 to about 100 ng/mL, from about 0.1 to about 50 ng/mL, from about 0.2 to about 1500 ng/mL, from about 0.2 to about 1000 ng/mL, from about 0.2 to about 900 ng/mL, from about 0.2 to about 800 ng/mL, from about 0.2 to about 700 ng/mL, from about 0.2 to about 600 ng/mL, from about 0.2 to about 500 ng/mL, from about 0.2 to about 400 ng/mL, from about 0.2 to about 300 ng/mL, from about 0.2 to about 200 ng/mL, from about 0.2 to about 100 ng/mL, from about 0.2 to about 50 ng/mL, from about 0.5 to about 1500 ng/mL, from about 0.5 to about 1000 ng/mL, from about 0.5 to about 900 ng/mL, from about 0.5 to about 800 ng/mL, from about 0.5 to about 700 ng/mL, from about 0.5 to about 600 ng/mL, from about 0.5 to about 500 ng/mL, from about 0.5 to about 400 ng/mL, from about 0.5 to about 300 ng/mL, from about 0.5 to about 200 ng/mL, from about 0.5 to about 100 ng/mL, from about 0.5 to about 50 ng/mL, from about 1 to about 1500 ng/mL, from about 1 to about 1000 ng/mL, from about 1 to about 900 ng/mL, from about 1 to about 800 ng/mL, from about 1 to about 700 ng/mL, from about 1 to about 600 ng/mL, from about 1 to about 500 ng/mL, from about 1 to about 400 ng/mL, from about 1 to about 300 ng/mL, from about 1 to about 200 ng/mL, from about 1 to about 100 ng/mL, or from about 1 to about 50 ng/mL.

In one aspect, the present disclosure provides a composition for detecting, measuring, or analyzing a sample using liquid chromatography described herein, wherein the composition comprises at least one solvent from the group consisting of trifluoroacetic acid, ammonia, formic acid, acetonitrile, methanol, water, and 2-propanol. In some embodiments, the composition comprises trifluoroacetic acid. In some embodiments, the composition comprises ammonia. In some embodiments, the composition comprises formic acid. In some embodiments, the composition comprises acetonitrile. In some embodiments, the composition comprises methanol. In some embodiments, the composition comprises water. In some embodiments, the composition comprises and 2-propanol.

In some embodiments, the composition comprises trifluoroacetic acid in a range from about 0.5% to about 5% v/v. In some embodiments, the composition further comprises acetonitrile, methanol, water, and 2-propanol. In some embodiments, the composition comprises equal volumes of acetonitrile, methanol, water, and 2-propanol.

In one aspect, the present disclosure provides a composition for detecting, measuring, or analyzing a sample using liquid chromatography described herein, wherein the composition comprises at least one solvent from the group consisting of N, N-dimethylsulfoxide (DMSO), acetonitrile, methanol, trifluoracetic acid, formic acid, ammonium, and water. In some embodiments, the composition comprises N, N-dimethylsulfoxide (DMSO). In some embodiments, the composition comprises acetonitrile. In some embodiments, the composition comprises methanol. In some embodiments, the composition comprises trifluoracetic acid. In some embodiments, the composition comprises formic acid. In some embodiments, the composition comprises ammonium. In some embodiments, the composition comprises water.

In some embodiments, the composition comprises DMSO in a range from about 1% to about 10% v/v. In some embodiments, the composition cleans a liquid chromatography autosampler components comprising at least one selected from the group consisting of needle, high pressure valve, injection port and stainless tubes used for the connections.

In some embodiments, the lower limit quality control (LLQC) of the method described herein comprises at least 0.01, 0.02, 0.03, 0.04, 0.05, 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or 1000 ng/mL. In some embodiments, the method achieves lower limit quality control (LLQC) of at most 10000, 9000, 8000, 7000, 6000, 5000, 4000, 3000, 2000, 1000, 950, 900, 850, 800, 750, 700, 650, 600, 550, 500, 450, 400, 350, 300, 250, 200, 150, 100, 90, 80, 70, 60, 50, 40, 30, 20, or 10 ng/mL. In some embodiment, the method achieves lower limit quality control (LLQC) from about 0.01 to about 1500 ng/mL, from about 0.01 to about 1000 ng/mL, from about 0.01 to about 900 ng/mL, from about 0.01 to about 800 ng/mL, from about 0.01 to about 700 ng/mL, from about 0.01 to about 600 ng/mL, from about 0.01 to about 500 ng/mL, from about 0.01 to about 400 ng/mL, from about 0.01 to about 300 ng/mL, from about 0.01 to about 200 ng/mL, from about 0.01 to about 100 ng/mL, from about 0.01 to about 50 ng/mL, from about 0.1 to about 1500 ng/mL, from about 0.1 to about 1000 ng/mL, from about 0.1 to about 900 ng/mL, from about 0.1 to about 800 ng/mL, from about 0.1 to about 700 ng/mL, from about 0.1 to about 600 ng/mL, from about 0.1 to about 500 ng/mL, from about 0.1 to about 400 ng/mL, from about 0.1 to about 300 ng/mL, from about 0.1 to about 200 ng/mL, from about 0.1 to about 100 ng/mL, from about 0.1 to about 50 ng/mL, from about 0.2 to about 1500 ng/mL, from about 0.2 to about 1000 ng/mL, from about 0.2 to about 900 ng/mL, from about 0.2 to about 800 ng/mL, from about 0.2 to about 700 ng/mL, from about 0.2 to about 600 ng/mL, from about 0.2 to about 500 ng/mL, from about 0.2 to about 400 ng/mL, from about 0.2 to about 300 ng/mL, from about 0.2 to about 200 ng/mL, from about 0.2 to about 100 ng/mL, from about 0.2 to about 50 ng/mL, from about 0.5 to about 1500 ng/mL, from about 0.5 to about 1000 ng/mL, from about 0.5 to about 900 ng/mL, from about 0.5 to about 800 ng/mL, from about 0.5 to about 700 ng/mL, from about 0.5 to about 600 ng/mL, from about 0.5 to about 500 ng/mL, from about 0.5 to about 400 ng/mL, from about 0.5 to about 300 ng/mL, from about 0.5 to about 200 ng/mL, from about 0.5 to about 100 ng/mL, from about 0.5 to about 50 ng/mL, from about 1 to about 1500 ng/mL, from about 1 to about 1000 ng/mL, from about 1 to about 900 ng/mL, from about 1 to about 800 ng/mL, from about 1 to about 700 ng/mL, from about 1 to about 600 ng/mL, from about 1 to about 500 ng/mL, from about 1 to about 400 ng/mL, from about 1 to about 300 ng/mL, from about 1 to about 200 ng/mL, from about 1 to about 100 ng/mL, or from about 1 to about 50 ng/mL.

In some embodiments, the upper limit quality control (ULQC) of the method described herein comprises at least 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, or 1000 ng/mL. In some embodiments, the method achieves upper limit quality control (ULQC) of at most 10000, 9000, 8000, 7000, 6000, 5000, 4000, 3000, 2000, 1000, 950, 900, 850, 800, 750, 700, 650, 600, 550, 500, 450, 400, 350, 300, 250, 200, 150, 100, 90, 80, 70, 60, 50, 40, 30, 20, or 10 ng/mL. In some embodiment, the method achieves upper limit quality control (ULQC) from about 1 to about 1500 ng/mL, from about 1 to about 1000 ng/mL, from about 1 to about 900 ng/mL, from about 1 to about 800 ng/mL, from about 1 to about 700 ng/mL, from about 1 to about 600 ng/mL, from about 1 to about 500 ng/mL, from about 1 to about 400 ng/mL, from about 1 to about 300 ng/mL, from about 1 to about 200 ng/mL, from about 1 to about 100 ng/mL, from about 1 to about 50 ng/mL, from about 2 to about 1500 ng/mL, from about 2 to about 1000 ng/mL, from about 2 to about 900 ng/mL, from about 2 to about 800 ng/mL, from about 2 to about 700 ng/mL, from about 2 to about 600 ng/mL, from about 2 to about 500 ng/mL, from about 2 to about 400 ng/mL, from about 2 to about 300 ng/mL, from about 2 to about 200 ng/mL, from about 2 to about 100 ng/mL, from about 2 to about 50 ng/mL, from about 5 to about 1500 ng/mL, from about 5 to about 1000 ng/mL, from about 5 to about 900 ng/mL, from about 5 to about 800 ng/mL, from about 5 to about 700 ng/mL, from about 5 to about 600 ng/mL, from about 5 to about 500 ng/mL, from about 5 to about 400 ng/mL, from about 5 to about 300 ng/mL, from about 5 to about 200 ng/mL, from about 5 to about 100 ng/mL, from about 5 to about 50 ng/mL, from about 10 to about 1500 ng/mL, from about 10 to about 1000 ng/mL, from about 10 to about 900 ng/mL, from about 10 to about 800 ng/mL, from about 10 to about 700 ng/mL, from about 10 to about 600 ng/mL, from about 10 to about 500 ng/mL, from about 10 to about 400 ng/mL, from about 10 to about 300 ng/mL, from about 10 to about 200 ng/mL, from about 10 to about 100 ng/mL, or from about 10 to about 50 ng/mL.

In some embodiments, a flow rate used in the method described herein may be 0.3 mL/min. In some embodiments, a flow rate may be at least 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, or 10 mL/min. In some embodiments, a flow rate may be at most 100, 95, 90, 85, 80, 75, 70, 65, 60, 55, 50, 45, 40, 35, 30, 25, 20, 15, 10, 5, 4, 3, 2, 1, 0.9, 0.8, 0.7, 0.6, 0.5, 0.4, 0.3, 0.2, 0.1, or 0.05 mL/min. In some embodiments, the flow rat may be in a range from about 0.05 to about 100, from about 0.05 to about 90, from about 0.05 to about 80, from about 0.05 to about 70, from about 0.05 to about 60, from about 0.05 to about 50, from about 0.05 to about 40, from about 0.05 to about 30, from about 0.05 to about 20, from about 0.05 to about 10, from about 0.1 to about 100, from about 0.1 to about 90, from about 0.1 to about 80, from about 0.1 to about 70, from about 0.1 to about 60, from about 0.1 to about 50, from about 0.1 to about 40, from about 0.1 to about 30, from about 0.1 to about 20, from about 0.1 to about 10, from about 0.1 to about 5, from about 0.1 to about 4, from about 0.1 to about 3, from about 0.1 to about 2, from about 0.1 to about 1, from about 0.1 to about 0.5, from about 0.1 to about 0.4, from about 0.1 to about 0.3, from about 0.1 to about 0.2, from about 0.2 to about 100, from about 0.2 to about 90, from about 0.2 to about 80, from about 0.2 to about 70, from about 0.2 to about 60, from about 0.2 to about 50, from about 0.2 to about 40, from about 0.2 to about 30, from about 0.2 to about 20, from about 0.2 to about 10, from about 0.2 to about 5, from about 0.2 to about 4, from about 0.2 to about 3, from about 0.2 to about 2, from about 0.2 to about 1, from about 0.2 to about 0.5, from about 0.2 to about 0.4, from about 0.2 to about 0.3, from about 0.3 to about 100, from about 0.3 to about 90, from about 0.3 to about 80, from about 0.3 to about 70, from about 0.3 to about 60, from about 0.3 to about 50, from about 0.3 to about 40, from about 0.3 to about 30, from about 0.3 to about 20, from about 0.3 to about 10, from about 0.3 to about 5, from about 0.3 to about 4, from about 0.3 to about 3, from about 0.3 to about 2, from about 0.3 to about 1, from about 0.3 to about 0.5, from about 0.3 to about 0.4, from about 0.4 to about 100, from about 0.4 to about 90, from about 0.4 to about 80, from about 0.4 to about 70, from about 0.4 to about 60, from about 0.4 to about 50, from about 0.4 to about 40, from about 0.4 to about 30, from about 0.4 to about 20, from about 0.4 to about 10, from about 0.4 to about 5, from about 0.4 to about 4, from about 0.4 to about 3, from about 0.4 to about 2, from about 0.4 to about 1, from about 0.4 to about 0.5, from about 0.5 to about 100, from about 0.5 to about 90, from about 0.5 to about 80, from about 0.5 to about 70, from about 0.5 to about 60, from about 0.5 to about 50, from about 0.5 to about 40, from about 0.5 to about 30, from about 0.5 to about 20, from about 0.5 to about 10, from about 0.5 to about 5, from about 0.5 to about 4, from about 0.5 to about 3, from about 0.5 to about 2, or from about 0.5 to about 1 mL/min.

In some embodiments, a volume of the sample injected into the liquid chromatography described herein may be about 25 μL. In some embodiments, an injection volume of analytical condition may be at least 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, or 1000 μL. In some embodiments, an injection volume of analytical condition comprises at most 10, 20, 30, 40, 50, 60, 70, 80, 90, 100, 200, 300, 400, 500, 600, 700, 800, 900, 1000, 1500, 2000, 2500, 3000, 3500, 4500, or 5000 μL. In some embodiments, an injection volume may be in a range from about 1 to about 5000, from about 1 to about 4000, from about 1 to about 3000, from about 1 to about 2000, from about 1 to about 1000, from about 1 to about 500, from about 1 to about 400, from about 1 to about 300, from about 1 to about 200, from about 1 to about 100, from about 1 to about 50, from about 1 to about 30, from about 5 to about 5000, from about 1 to about 4000, from about 5 to about 3000, from about 5 to about 2000, from about 5 to about 1000, from about 5 to about 500, from about 5 to about 400, from about 5 to about 300, from about 5 to about 200, from about 5 to about 100, from about 5 to about 50, from about 5 to about 30, from about 10 to about 5000, from about 10 to about 4000, from about 10 to about 3000, from about 10 to about 2000, from about 10 to about 1000, from about 10 to about 500, from about 10 to about 400, from about 10 to about 300, from about 10 to about 200, from about 10 to about 100, from about 10 to about 50, from about 10 to about 40, from about 10 to about 30, from about 10 to about 20, from about 20 to about 5000, from about 20 to about 4000, from about 20 to about 3000, from about 20 to about 2000, from about 20 to about 1000, from about 20 to about 500, from about 20 to about 400, from about 20 to about 300, from about 20 to about 200, from about 20 to about 100, from about 20 to about 50, from about 20 to about 40, from about 20 to about 30, from about 25 to about 5000, from about 25 to about 4000, from about 25 to about 3000, from about 25 to about 2000, from about 25 to about 1000, from about 25 to about 500, from about 25 to about 400, from about 25 to about 300, from about 25 to about 200, from about 25 to about 100, from about 25 to about 50, from about 25 to about 40, or from about 25 to about 30 μL.

In some embodiments, an accuracy of the method described herein may be at least 50, 55, 60, 65, 70, 75, 80, 85, 90, 95, 100, 105, 110, 115, or 120%. In some embodiments, an accuracy may be at most 85, 86, 87, 88, 89, 90, 91, 92, 93, 94, 95, 96, 97, 98, 99, 100, 105, 110, 115, 120, 125, 130, 135, 140, 145, or 150%. In some embodiments, an accuracy may be in a range from about 75 to about 150, from about 75 to about 140, from about 75 to about 130, from about 75 to about 120, from about 75 to about 115, from about 75 to about 110, from about 75 to about 105, from about 75 to about 100, from about 75 to about 95, from about 80 to about 150, from about 80 to about 140, from about 80 to about 130, from about 80 to about 120, from about 80 to about 115, from about 80 to about 110, from about 80 to about 105, from about 80 to about 100, from about 80 to about 90, from about 80 to about 95, from about 85 to about 140, from about 85 to about 130, from about 85 to about 120, from about 85 to about 115, from about 85 to about 110, from about 85 to about 105, from about 85 to about 100, from about 85 to about 95, from about 85 to about 90, from about 90 to about 150, from about 90 to about 140, from about 90 to about 130, from about 90 to about 120, from about 90 to about 115, from about 90 to about 110, from about 90 to about 105, from about 90 to about 100, from about 90 to about 95, from about 95 to about 140, from about 95 to about 130, from about 95 to about 120, from about 95 to about 115, from about 95 to about 110, from about 95 to about 105, from about 95 to about 100%.

In some embodiments, a precision (relative standard deviation (RSD) value of the method described herein is within about 15%. In some embodiments, a precision value of the method described herein may be at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 20, 25, or 30%. In some embodiments, a precision (relative standard deviation (RSD) value may be at most about 4, 4.5, 5, 5.5, 6, 6.5, 7, 7.5, 8, 8.5, 9, 9.5, 10, 11, 12, 13, 14, 15, 20, 25, 30, 35, 40, 45, or 50%. In some embodiments, a precision (relative standard deviation (RSD)) value may be in a range from about 1 to about 20, from about 1 to about 15, from about 1 to about 14, from about 1 to about 13, from about 1 to about 12, from about 1 to about 11, from about 1 to about 10, from about 1 to about 9, from about 1 to about 8, from about 1 to about 7, from about 1 to about 6, from about 1 to about 5, from about 1 to about 4, from about 1 to about 3, from about 1 to about 2, from about 2 to about 20, from about 2 to about 15, from about 2 to about 14, from about 2 to about 13, from about 2 to about 12, from about 2 to about 11, from about 2 to about 10, from about 2 to about 9, from about 2 to about 8, from about 2 to about 7, from about 2 to about 6, from about 2 to about 5, from about 2 to about 4, from about 2 to about 3, from about 4 to about 15, from about 4 to about 14, from about 4 to about 13, from about 4 to about 12, from about 4 to about 11, from about 4 to about 10, from about 4 to about 9, from about 4 to about 8, from about 4 to about 7, from about 4 to about 6, from about 4 to about 5, from about 6 to about 15, from about 6 to about 14, from about 6 to about 13, from about 6 to about 12, from about 6 to about 11, from about 6 to about 10, from about 6 to about 9, from about 6 to about 8, or from about 6 to about 7%.

Hereinafter, the present disclosure will be described in more detail with reference to the following examples. But the following Examples are intended to illustrate the present embodiments, and the scope of the Examples is not limited thereto only.

### EXAMPLES

In the example, two solvent mixtures are developed and used for LC-MS/MS analysis, each of the mixtures having multiple solvents in an optimized proportion, specifically designed to get rid of the contamination from HPLC autosampler parts. These two solvent mixtures are being employed in series as per a pre-and post-treatment program using an autosampler multi-rinse feature. These solvent mixtures are used to clean the HPLC autosampler components e.g. needle, high pressure valve, injection port and stainless tubes used for the connections. Additionally, any potential non-specific adsorption is being taken care by utilizing a completely bioinert HPLC column having a proprietary bioinert coating inside the ss column body.

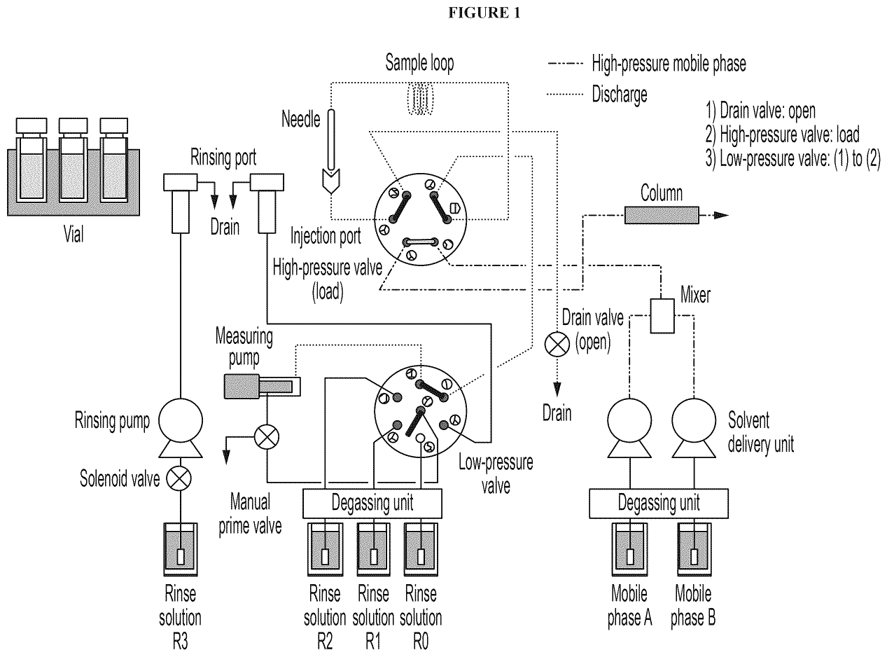

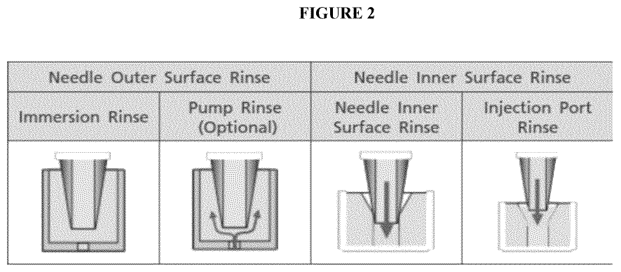

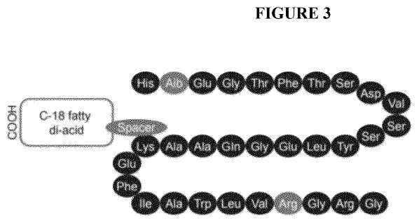

Diagram of internal rinsing of needle with rinse solution is shown in FIG. 1 and diagram of internal and external rinsing is shown in FIG. 2.

Semaglutide is a peptide used as an antidiabetic medication for the treatment of type-2 diabetes. It is also used as an anti-obesity medication for weight loss. Recently, studies have shown that Semaglutide also works on the brain, suggesting its potential utility for various diseases, including Parkinson's disease and Alzheimer's disease. To overcome these challenges an attempt was to develop an MRM based LC-MS/MS method with low limit of quantification (LLOQ) level, no carryover and wide dynamic range, ideal for Pharmacokinetics study of Semaglutide in human plasma. Shimadzu LCMS-8060NX was used to determine Semaglutide in plasma at low levels.

Human plasma was procured from local vendor to prepare calibration standards and quality control (QC) samples. Precursor ion selection, MRM optimization at different collision energies and voltages was done using Shimadzu's “Optimization for method” tool. Optimized MRM for 2 product ions with optimized voltages and collision energies (CE) were developed.

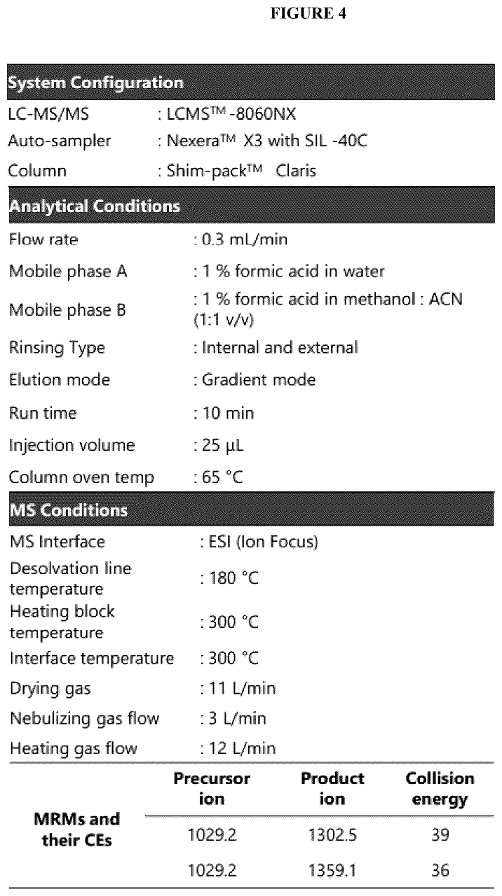

A LC method (FIG. 4) was developed using UHPLC column (Shim-pack Claris) to elute Semaglutide with no carry over. Using the developed LC method and optimized MRM, LLOQ of 0.2 ng/ml and upper limit of quantification (ULOQ) of 600 ng/ml was achieved with no carry over.

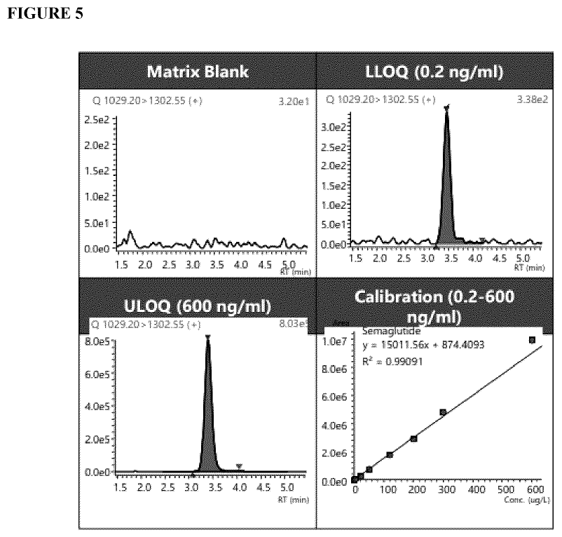

For Quantitation, a wide linearity batch ranging from 0.2 to 600 ng/ml was processed in human plasma. For QC check lower limit quality control (LLQC), lower quality control (LQC), medium quality control (MQC) and higher quality control (HQC) samples were processed in replicates and were quantified against the linearity. The accuracy for the calibration standards and QC samples was found to be within acceptable range (FIG. 5).

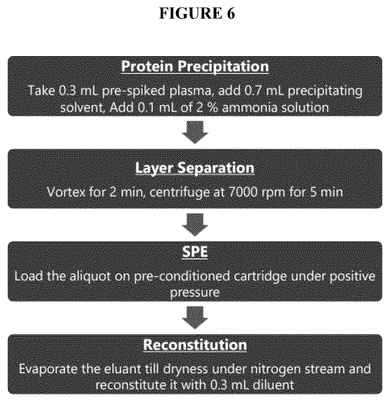

Schematic diagram of a sample analysis is shown in FIG. 6. 0.3 mL of pre-spiked plasma with 0.7 mL precipitating solvent and 0.1 mL of 2% ammonia solution were vortexed for 2 minutes and centrifuged at 7000 rpm for 5 minutes. Then samples were loaded on a pre-conditioned cartridge under positive pressure. Evaporated eluant were reconstituted with 0.3 mL diluent.

Validation parameters like specificity, linearity, accuracy, precision and carryover were studied as per ICH M10 Guidelines.

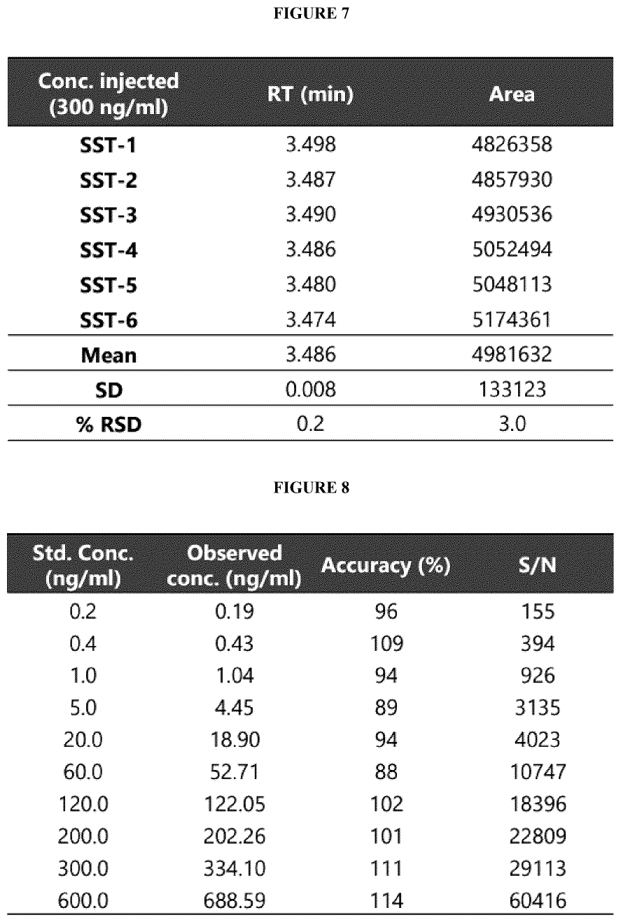

System precision: System precision was evaluated by calculating variation of the peak area and retention time (RT) of six replicates of 300 ng/ml processed Semaglutide standard. The % RSD was found to be less than 5 for peak area, whereas the difference in RTs for 6 replicate injections was found to be within +0.1 min. Specificity of the method was determined by comparing the response of blank sample (reagent and matrix) against reporting level. Response in reagent/matrix blank sample was well within <20% of the reporting limit and met the acceptance criteria (FIG. 7).

Linearity study: For linearity study, processed calibration standards were used. All calibration standards were found within 85 to 115% accuracy (FIG. 8). The linearity is shown in FIG. 8.

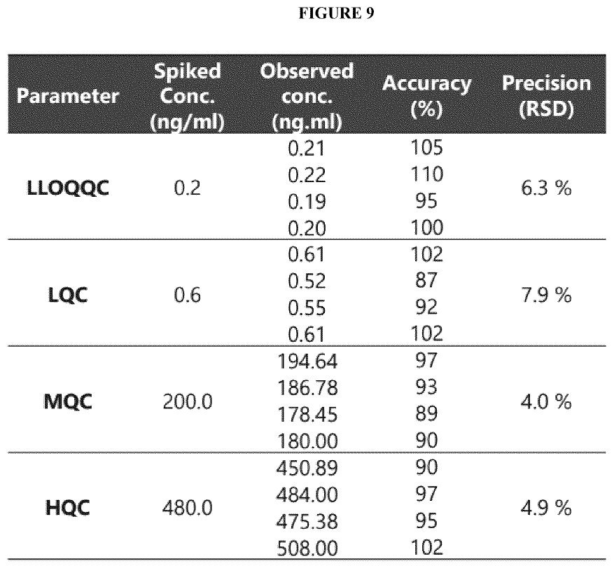

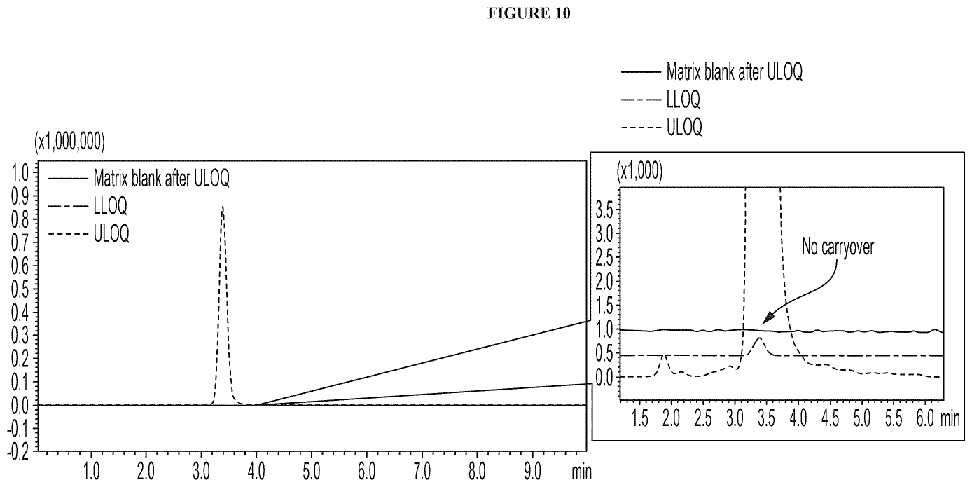

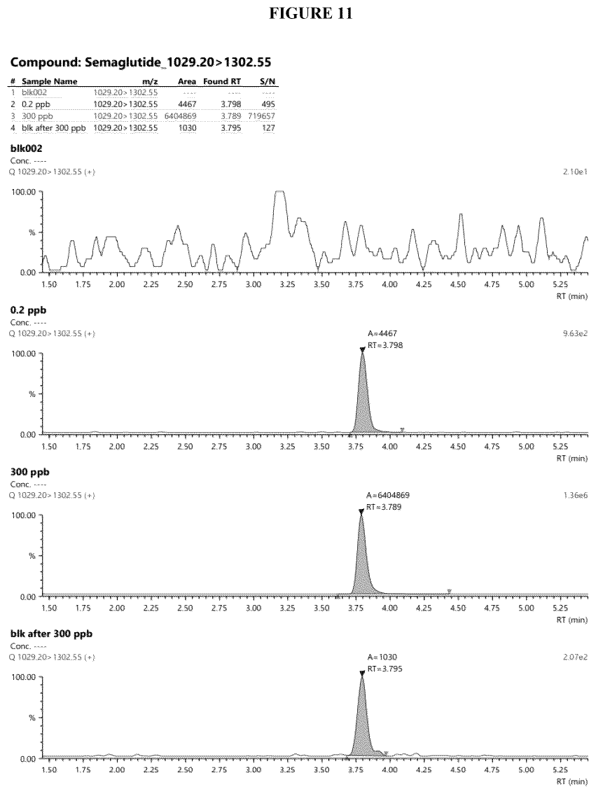

Accuracy and Precision study: QC samples at 4 different levels LLQC, LQC, MQC and HQC were processed in replicates and quantified for accuracy and precision study. The observed results were within acceptance criteria of % RSD±15% (FIG. 9).

Carryover: Carryover was assessed by analysing blank sample after injecting highest calibration standard, the area response at the retention time of Semaglutide for the blank sample analysed after highest calibration standard was found to be less than 20.0% of the area response of the LLOQ standard (FIG. 10).

These experimental date shows a highly sensitive and precise method for quantifying GLP-1 peptide in human plasma using the Shimadzu LCMS-8060NX system. This method addresses common challenges, including achieving low-level LLOQ, wide dynamic range, and carryover-free detection. Moreover, the results met the accuracy and precision standards of ICH M10 guidelines, confirming the reliability of the method.

FIG. 11 shows a result of a blank analysis performed before analyzing Semaglutide. A blank analysis (BLK) was an analysis performed without injecting a sample. Normally, the signal intensity should be 0 in a blank analysis, but due to noise, a very small signal was generated as shown in the FIG. 11 (BLK002).

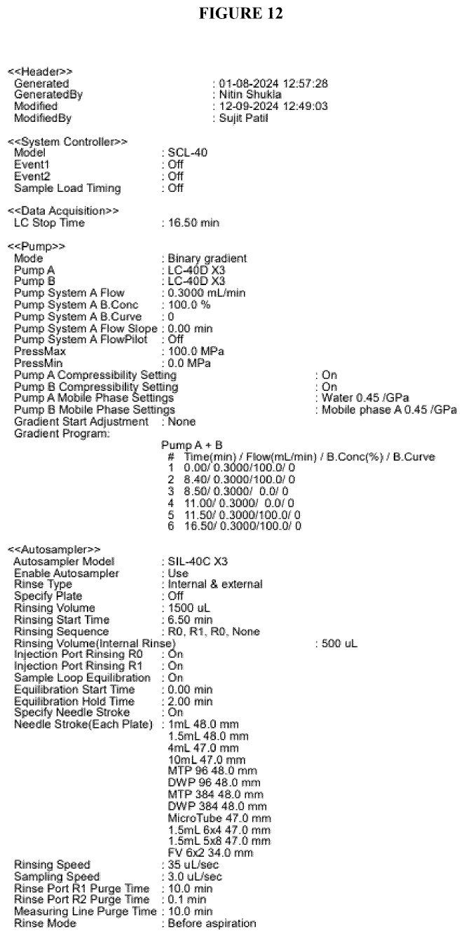

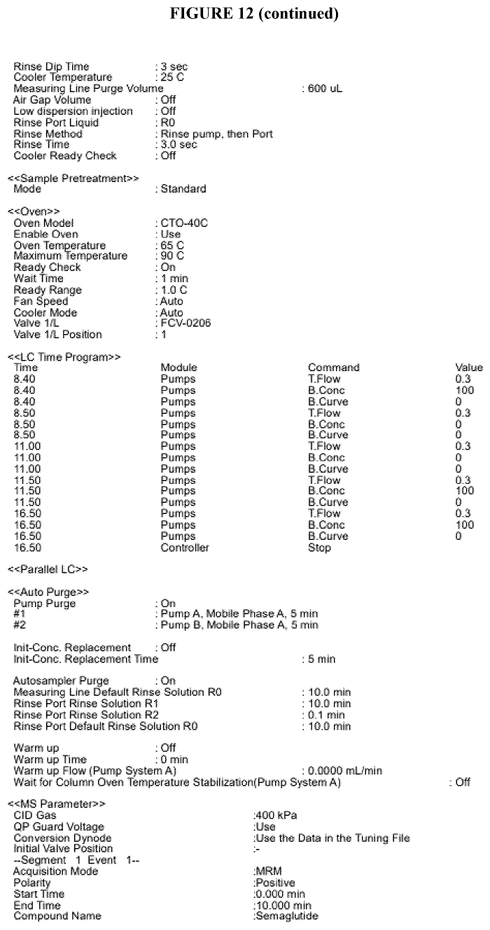

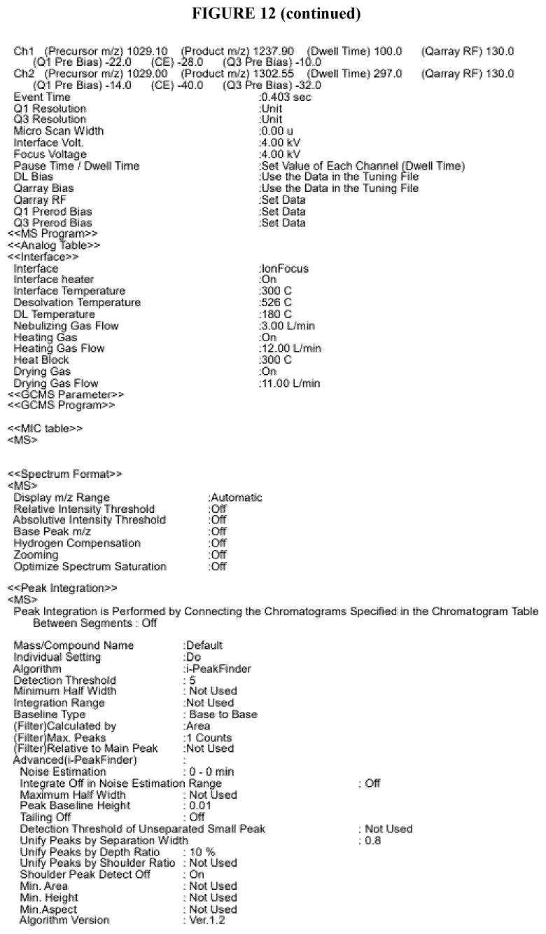

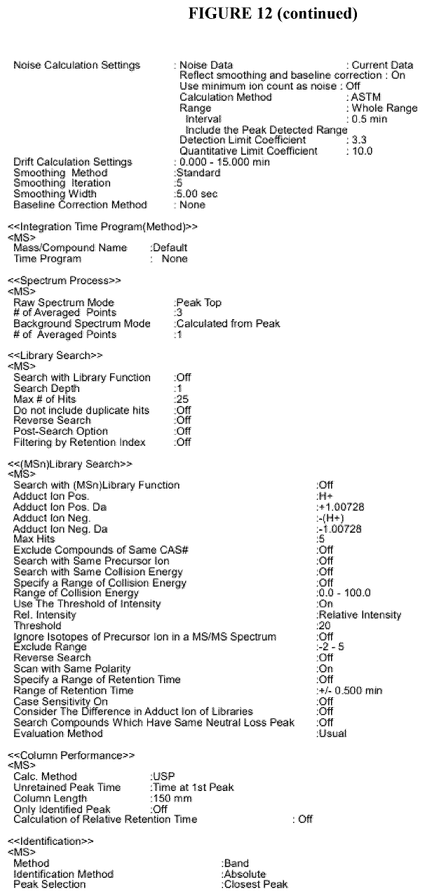

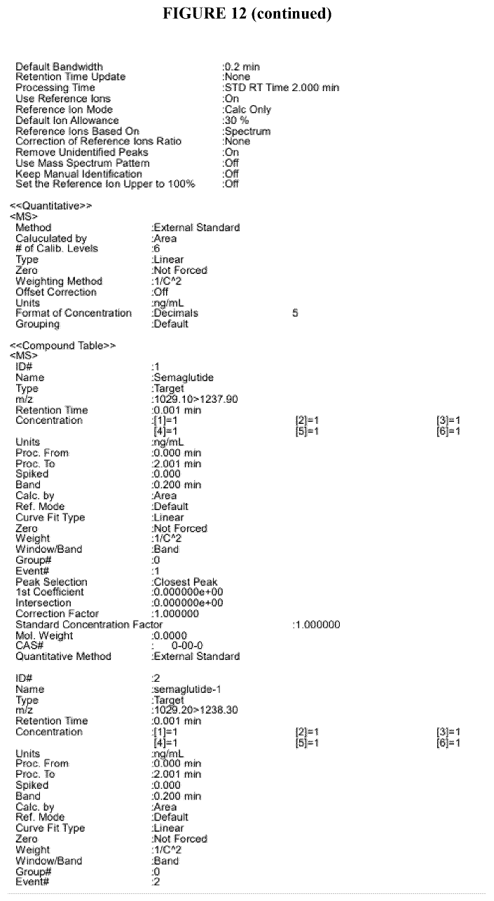

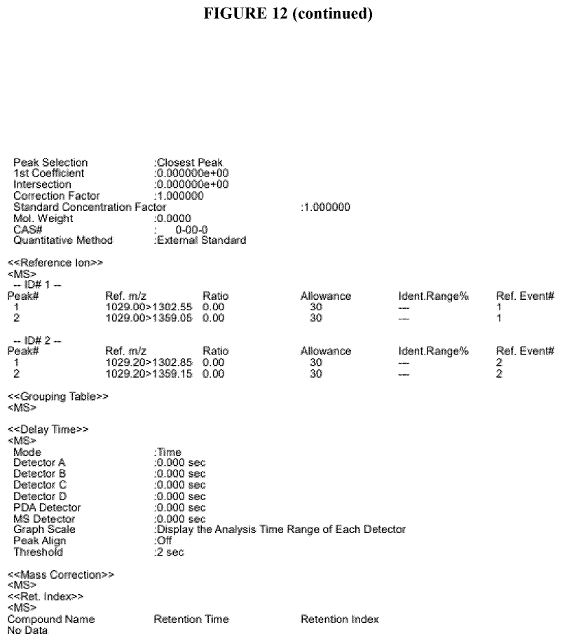

FIG. 12 shows the detailed method used in the example.

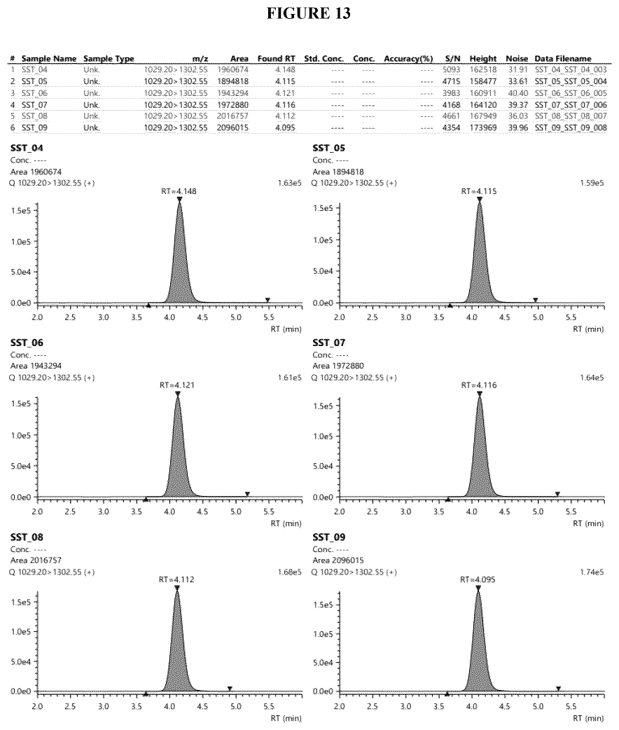

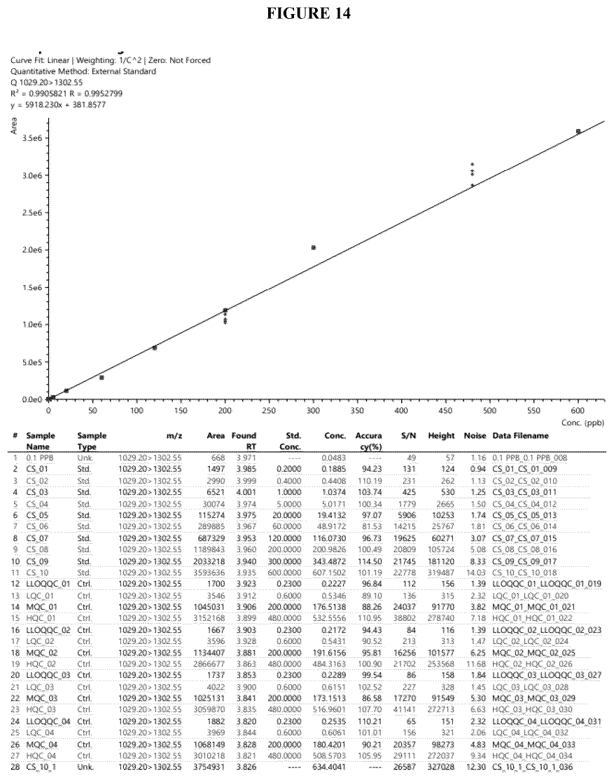

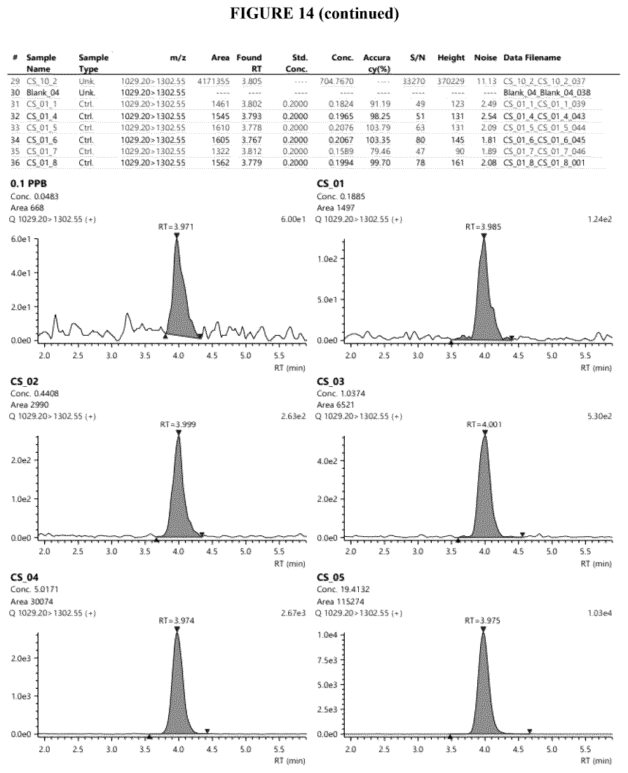

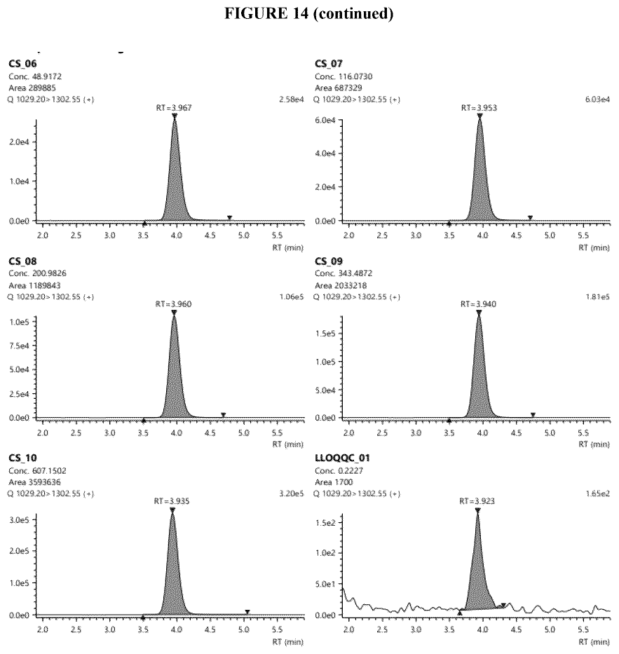

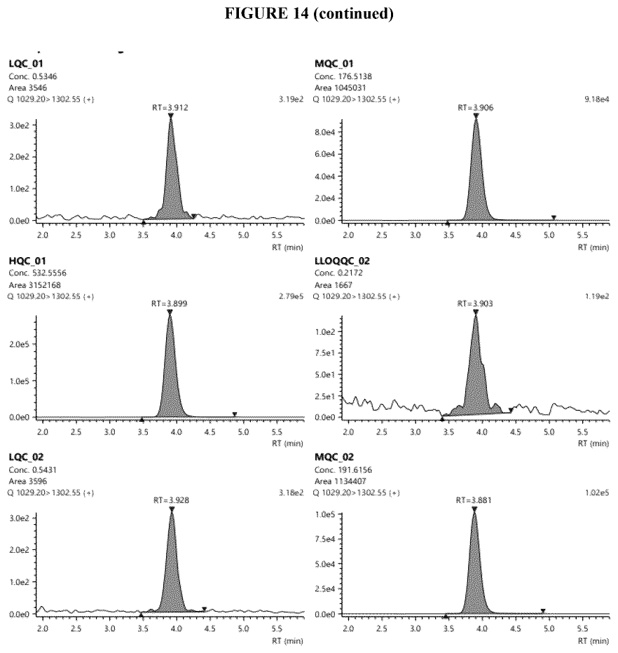

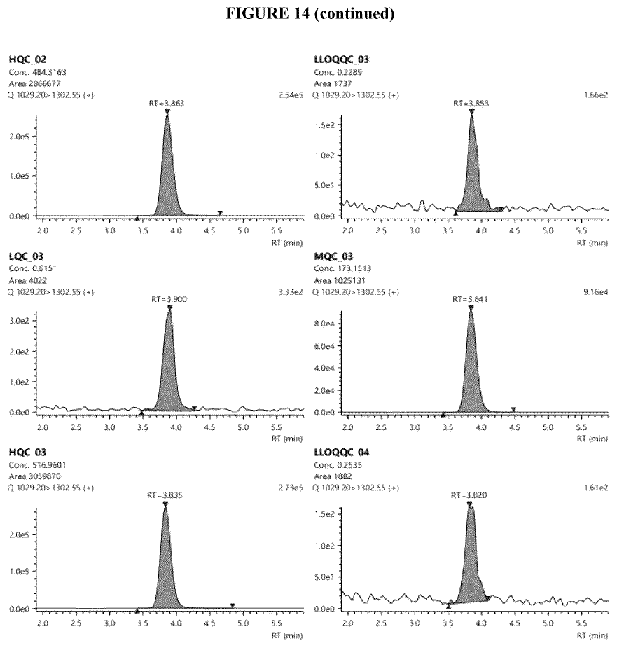

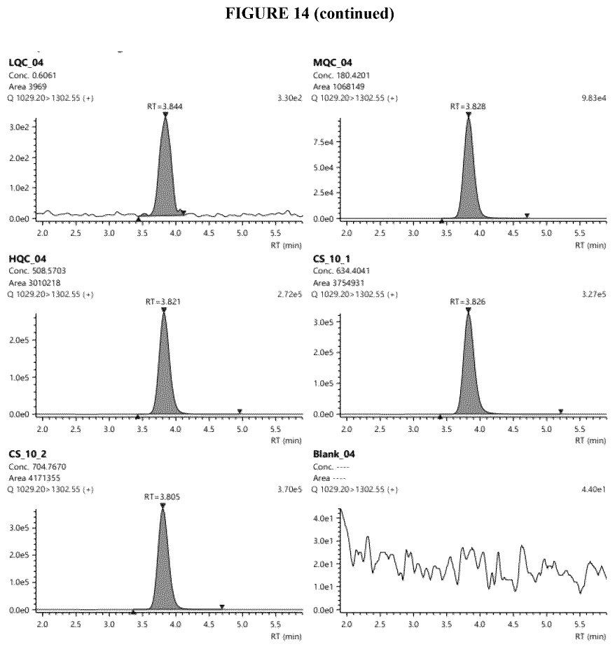

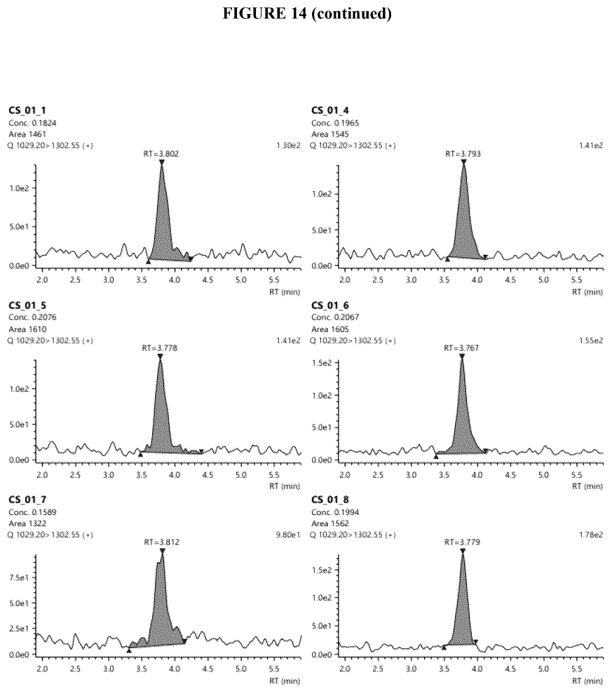

These examples allow us to quantitatively analyze semaglutide with excellent accuracy due to no carryover even after analyzing high concentration quality check human plasma sample. These examples allow us to quantitatively analyze semaglutide with a range of 0.2 ng/mL to 600 ng/mL in biological matrix such as human plasma. Multi-rinse autosampler option/settings, an optimized rinsing solution with program and a proprietary bioinert column are the minimum features that required to have a sensitive and accurate quantitative analysis of synthetic peptide (FIGS. 13 and 14).

### Exemplary Embodiments

Embodiment 1. A method of separating, purifying, detecting, measuring, or analyzing a sample using liquid chromatography comprising rinsing a sampling means of a liquid chromatography system with a first rinsing solution.

Embodiment 2. The method according to embodiment 1, wherein the first rinsing solution comprises two or more solvents from the group consisting of trifluoroacetic acid, ammonia, formic acid, acetonitrile, methanol, water, and 2-propanol.

Embodiment 3. The method according to embodiment 1 or embodiment 2, wherein the first rinsing solution comprises trifluoroacetic acid.

Embodiment 4. The method according to any one of the preceding embodiments, wherein the first rinsing solution comprises trifluoroacetic acid in a range from about 0.5% to about 5% v/v.

Embodiment 5. The method according to any one of the preceding embodiments, wherein the first rinsing solution comprises 1% v/v trifluoroacetic acid.

Embodiment 6. The method according to any one of the preceding embodiments, wherein the first rinsing solution further comprises at least one

selected from the group consisting of acetonitrile, methanol, water, and 2-propanol. Embodiment 7. The method according to any one of the preceding embodiments, wherein the first rinsing solution further comprises acetonitrile, methanol, water, and 2-propanol.

Embodiment 8. The method according to any one of the preceding embodiments, wherein the first rinsing solution comprises equal volumes of acetonitrile, methanol, water, and 2-propanol.

Embodiment 9. The method according to any one of the preceding embodiments, wherein the rinsing the sampling means with a first rinsing solution comprises purging the first rinsing solution from the sampling means.

Embodiment 10. The method according to any one of the preceding embodiments, wherein the rinsing the sampling means with the first rinsing solution further comprises rinsing an injection port.

Embodiment 11. The method according to any one of the preceding embodiments, wherein the method further comprises rinsing the sampling means with a second rinsing solution.

Embodiment 12. The method according to any one of the preceding embodiments, wherein the second rinsing solution comprises one or more solvents selected from the group consisting of N, N-dimethylsulfoxide (DMSO), acetonitrile, methanol, trifluoracetic acid, formic acid, ammonium, and water,

Embodiment 13. The method according to any one of the preceding embodiments, wherein the second rinsing solution comprises DMSO in a range from about 1% to about 10% v/v.

Embodiment 14. The method according to any one of the preceding embodiments, wherein the second rinsing solution comprises DMSO in a range from about 4% to about 6% v/v.

Embodiment 15. The method according to any one of the preceding embodiments, wherein the second rinsing solution comprises 5% v/v DMSO.

Embodiment 16. The method according to any one of the preceding embodiments, wherein the rinsing the sampling means with the second rinsing solution comprises immersing a portion of the sampling means in the second rinsing solution.

Embodiment 17. The method according to any one of the preceding embodiments, wherein the rinsing the sampling means with the second rinsing solution further comprises agitating or pumping the second rinsing solution while the portion of the sampling means remain immersed.

Embodiment 18. The method according to any one of the preceding embodiments, wherein the rinsing the sampling means comprises cleaning a liquid chromatography autosampler components.

Embodiment 19. The method according to embodiment 18, wherein the autosampler component comprises at least one selected from the group consisting of needle, high pressure valve, injection port and stainless tubes used for the connections.

Embodiment 20. The method according to any one of the preceding embodiments, wherein the method further comprises loading a liquid chromatography column with the sample via the sampling means and/or performing liquid chromatography with the liquid chromatography column.

Embodiment 21. The method according to any one of the preceding embodiments, wherein the method further comprises performing mass spectrometry on an elute from the liquid chromatography.

Embodiment 22. The method according to any one of the preceding embodiments, wherein the sample comprises a peptide.

Embodiment 23. The method according to any one of the preceding embodiments, wherein the sample comprises a synthetic peptide.

Embodiment 24. The method according to any one of the preceding embodiments, wherein the sample comprises semaglutide.

Embodiment 25. The method according to any one of the preceding embodiments, wherein the liquid chromatography comprises high-performance liquid chromatography (HPLC).

Embodiment 26. The method according to any one of the preceding embodiments, wherein the liquid chromatography comprises fast protein liquid chromatography (FPLC).

Embodiment 27. The method according to any one of the preceding embodiments, wherein the liquid chromatography comprises liquid-liquid chromatography.

Embodiment 28. The method according to any one of the preceding embodiments, wherein a column conditioning solution comprises one or more solvents selected from the group consisting of N, N-dimethylsulfoxide (DMSO), acetonitrile, methanol and formic acid.

Embodiment 29. The method according to any one of the preceding embodiments, wherein the column conditioning solution comprises from about 0.1% to about 5% v/v formic acid.

Embodiment 30. The method according to any one of the preceding embodiments, wherein the column conditioning solution comprises 0.5% v/v formic acid.

Embodiment 31. The method according to any one of the preceding embodiments, wherein the column conditioning solution comprises from about 0.1% to about 10% v/v N, N-dimethylsulfoxide (DMSO).

Embodiment 32. The method according to any one of the preceding embodiments, wherein the column conditioning solution comprises 1.0% v/v N, N-Dimethylsulfoxide (dmso).

Embodiment 33. The method according to any one of the preceding embodiments, wherein the method comprises separating, purifying, detecting, measuring or anlayzing a protein from the sample.

Embodiment 34. The method according to any one of the preceding embodiments, wherein the method comprises the separating or purifying.

Embodiment 35. The method according to any one of the preceding embodiments, wherein the method comprises the detecting, measuring or analyzing.

Embodiment 36. The method according to any one of the preceding embodiments, wherein the method comprises detecting, measuring or analyzing an amount, a concentration and/or mass of a protein in the sample.

Embodiment 37. The method according to any one of the preceding embodiments, wherein the protein is semaglutide.

Embodiment 38. A composition for detecting, measuring, or analyzing a sample using liquid chromatography according to any one of the preceding claims, wherein the composition comprises at least one solvent from the group consisting of trifluoroacetic acid, ammonia, formic acid, acetonitrile, methanol, water, and 2-propanol.

Embodiment 39. The composition according to embodiment 38 wherein the composition comprises trifluoroacetic acid in a range from about 0.5% to about 5% v /v.

Embodiment 40. The composition according to embodiment 38 or 39, wherein the composition further comprises acetonitrile, methanol, water, and 2-propanol.

Embodiment 41. The composition according to any one of the embodiments 38-40, wherein the composition comprises equal volumes of acetonitrile, methanol, water, and 2-propanol.

Embodiment 42. A composition for detecting, measuring, or analyzing a sample using liquid chromatography according to any one of the embodiments 1 to 37, wherein the composition comprises at least one solvent from the group consisting of N, N-dimethylsulfoxide (DMSO), acetonitrile, methanol, trifluoracetic acid, formic acid, ammonium, and water.

Embodiment 43. The composition according to embodiment 42, wherein the composition comprises DMSO in a range from about 1% to about 10% v/v.

Embodiment 44. The composition according to any one of embodiments 42 or 43, wherein the composition cleans a liquid chromatography autosampler components comprising at least one selected from the group consisting of needle, high pressure valve, injection port and stainless tubes used for the connections.

We claim:

1. A method of detecting, measuring, or analyzing a sample using a liquid using liquid chromatography, comprising;
a first rinsing step comprising rinsing inside of a sampling means of a liquid chromatography system with a first rinsing solution, and a second rinsing step comprising rinsing outside of a sampling means of a liquid chromatography system with a second rinsing solution.

2. The method according to claim 1, wherein the first rinsing solution comprises two or more solvents from the group consisting of trifluoroacetic acid, ammonia, formic acid, acetonitrile, methanol, water, and 2-propanol.

3. The method according to claim 1, wherein the first rinsing step comprises purging the first rinsing solution from the sampling means.

4. The method according to claim 1, wherein the rinsing inside of the sampling means with the first rinsing solution further comprises rinsing an injection port.

5. The method according to claim 1, wherein the second rinsing solution comprises one or more solvents selected from the group consisting of N, N-dimethylsulfoxide (DMSO), acetonitrile, methanol, trifluoracetic acid, formic acid, ammonium, and water.

6. The method according to claim 1, wherein the second rinsing solution comprises DMSO in a range from about 1% to about 10% v/v.

7. The method according to claim 1, wherein the rinsing outside of the sampling means with the second rinsing solution comprises immersing a portion of the sampling means in the second rinsing solution.

8. The method according to claim 1, wherein the rinsing outside of the sampling means with the second rinsing solution further comprises agitating or pumping the second rinsing solution while the portion of the sampling means remain immersed.

9. The method according to claim 1, wherein the sample comprises a peptide.

10. The method according to claim 1, wherein the sample comprises Semaglutide.

11. The method according to claim 1, wherein a column conditioning solution comprises one or more solvents selected from the group consisting of N, N-dimethylsulfoxide (DMSO), acetonitrile, methanol and formic acid.

12. The method according to claim 1, wherein the column conditioning solution comprises from about 0.1% to about 5% v/v formic acid.

13. The method according to claim 1, wherein the column conditioning solution comprises from about 0.1% to about 10% v/v N, N-dimethylsulfoxide (DMSO).
